SEQUENCE LISTING (8 sequences)

SEQ ID NO: 1 - DNA, 1671 bp, Artificial Sequence
  Description of Artificial Sequence Synthetic polynucleotide
atgctgctgctggtcacttctctgctgctgtgcgaactgccccaccccgcctttctgctgattcccgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagaacagaaactgatttccgaggaagatctgttcgtccccgtgttcctgcctgccaagccaacaactacccctgctccacgaccacctactccagcacctaccatcgcaagtcagcccctgtcactgcgacctgaggcttgccggccagcagctggaggagcagtgcacacccgaggcctggacttcgcatgcgatatctacatttgggcaccactggctggaacctgtggggtcctgctgctgagcctggtcatcaccctgtattgtaaccacagaaataggagcaaacgctcccgactgctgcattccgactacatgaacatgacacctcggagaccaggccccactagaaagcattaccagccatatgccccacccagggatttcgcagcctatcggagccggttcagcgtcgtgaaaagggggcgcaagaaactgctgtacatcttcaagcagccttttatgcgcccagtgcagacaactcaggaggaagacggatgctcttgtcggttcccagaggaggaggaaggaggctgcgagctgagagtgaagttcagccggagcgccgatgcaccagcatatcagcagggacagaatcagctgtacaacgagctgaatctgggcaggcgcgaggaatatgacgtgctggataagcgacgaggacgggaccccgaaatgggaggaaaacccagaaggaagaaccctcaggaggggctgtataatgaactgcagaaagacaagatggctgaggcatacagcgaaattggaatgaaaggagagcgccgacgggggaagggacacgatgggctgtaccagggactgtcaaccgccactaaagatacctacgacgcactgcacatgcaggctctgcccccaaga

SEQ ID NO: 2 - DNA, 1512 bp, Artificial Sequence
  Description of Artificial Sequence Synthetic polynucleotide
atgtacaggatgcaactcctgtcttgcattgcactaagtcttgcacttgtcacgaattcggcttgtattcttgatttgccgaagttttgcggtggaggttcggctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggctccaccagcggcagcggcaagccaggctccggcgaaggcagcaccaaaggcttctgggtcctggtcgtggtggggggggtgttggcctgttatagcctgctggtgactgtcgccttcattatattttgggtgagatccaaaagatcccggctgctccattctgactacatgaacatgactccaaggcgaccaggacctactagaaagcattatcagccgtacgctccaccccgggattttgcggcttatcgaagtagggaccagagattgccccctgacgcccacaagcctcccggggggggaagcttccgcacccccatccaggaagaacaagcagatgcacatagtactctggccaaaataagagtgaagttttctagaagcgcagacgcaccagcttaccagcagggccaaaatcagctgtacaacgagctgaacctgggccggagggaggaatacgatgtgctggataagcgccggggccgcgatcccgaaatgggcggcaagccacggcgaaagaatcctcaggagggactgtacaacgaactccaaaaggacaagatggcggaggcatactccgagatcggcatgaagggggaaagacggcggggcaaggggcatgacggtctgtatcaagggttgagtaccgccacaaaggatacctacgacgcccttcacatgcaggccctgccccctcgctagtaa

SEQ ID NO: 3 - protein, 557 aa, Artificial Sequence
  Description of Artificial Sequence Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEQKLISEEDLFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 502 aa, Artificial Sequence
  Description of Artificial Sequence Synthetic polypeptide
MYRMQLLSCIALSLALVTNSACILDLPKFCGGGSASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSTSGSGKPGSGEGSTKGFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 1401 bp, Artificial Sequence
  Description of Artificial Sequence Synthetic polynucleotide
atgcaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtgtaggcggttcaggcggcggtggctctggcggtggcggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtggttaccctctcacgttcggtgctgggacaaagttggaaataaaatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - protein, 466 aa, Artificial Sequence
  Description of Artificial Sequence Synthetic polypeptide
MQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 16 aa, Artificial Sequence
  Description of Artificial Sequence Synthetic peptide
MLWRRKIGPQMTLSAG

SEQ ID NO: 8 - protein, 222 aa, Artificial Sequence
  Description of Artificial Sequence Synthetic polypeptide
MMSETAPLAPTIPAPAEKTPVKKKAKKAGATAGKRKASGPPVSELITKAVAASKERSGVSLAALKKALAAAGYDVEKNNSRIKLGLKSLVSKGTLVQTKGTGASGSFKLNKKAASGEGKPKAKKAGAAKPRKPAGAAKKPKKVAGAATPKKSIKKTPKKVKKPATAAGTKKVAKSAKKVKTPQPKKAAKSPAKAKAPKPKAAKPKSGKPKVTKAKKAAPKKK

(19) United States
(12) Patent Application Publication
(10) Pub. No.: US 2021/0069242 A1
(43) Pub. Date: Mar. 11, 2021

(54) REPROGRAMMING OF POLYMORPHONUCLEAR LEUKOCYTES

(71) Applicants: Dmitry Dmitrievich GENKIN, St. Petersburg (RU); Alexey Vyacheslavovich Stepanov, Moscow (RU); Michael Vladimirovich Dubina, St. Petersburg (RU); Alexey Alexandrovich Bogdanov, St. Petersburg (RU); Alexander Gabibovich Gabibov, Moscow (RU)

(72) Inventors: Dmitry Dmitrievich GENKIN, St. Petersburg (RU); Alexey Vyacheslavovich Stepanov, Moscow (RU); Michael Vladimirovich Dubina, St. Petersburg (RU); Alexey Alexandrovich Bogdanov, St. Petersburg (RU); Alexander Gabibovich Gabibov, Moscow (RU)

(21) Appl. No.: 16/851,725

(22) Filed: Apr. 17, 2020

Related U.S. Application Data
(60) Provisional application No. 62/835,785, filed on Apr. 18, 2019.

(51) Int. Cl. A61K 35/17; C07K 14/725

(52) U.S. Cl. CPC A61K 35/17; C07K 14/7051

(57) ABSTRACT

The application is directed a method of treating a cancer in subject by administering to the subject a therapeutically effective amount of polymorphonuclear leukocytes genetically modified to express a recombinant chimeric antigen receptor (CAR) or T cell receptor (TCR). Further provided are modified polymorphonuclear leukocytes and related compositions for use in such methods.

### FIELD OF THE INVENTION

This application relates to methods and compositions for reprograming polymorphonuclear leukocytes for cancer immunotherapy.

### BACKGROUND

Cancer remains one of the top two causes of mortality in the United states, resulting in over 500,000 deaths per year. Immunotherapy with chimeric antigen receptor T (CAR-T) cells has demonstrated promise in the treatment of several hematological malignancies as well as some solid tumors. However, the efficacy and safety of this therapy are yet to be improved.

Polymorphonuclear leukocytes, or PMNs, are the most abundant circulating immune cells. They represent the first-line defense against infections and are potent effectors of inflammation. In addition, they release soluble chemotactic factors which guide the recruitment of both nonspecific and specific immune effector cells. The function of polymorphonuclear leukocytes provides possibilities for their clinical application in the treatment of cancer.

### SUMMARY OF THE INVENTION

The present invention provides improved cancer immunotherapies utilizing polymorphonuclear leukocytes (PMNs).

In one aspect, provided herein is a method of treating a cancer in a subject in need thereof comprising administering to the subject a therapeutically effective amount of polymorphonuclear leukocytes, wherein the polymorphonuclear leukocytes are genetically modified to express a recombinant chimeric antigen receptor (CAR) or T cell receptor (TCR). In some embodiments, the polymorphonuclear leukocytes express a chimeric antigen receptor (CAR) targeting 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDIL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

In some embodiments, the polymorphonuclear leukocytes express a chimeric antigen receptor (CAR) targeting CD19. In some embodiments, the CD19-targeting CAR comprises the amino acid sequence of SEQ ID NO: 3. In some embodiments, the CD19-targeting CAR is encoded by the nucleotide sequence of SEQ ID NO: 1.

In some embodiments, the polymorphonuclear leukocytes express a chimeric antigen receptor (CAR) targeting a human B cell receptor (BCR). In some embodiments, the BCR-targeting CAR comprises the amino acid sequence of SEQ ID NO: 4. In some embodiments, the BCR-targeting CAR is encoded by the nucleotide sequence of SEQ ID NO: 2.

In some embodiments, the CAR or the TCR targets a tumor-specific peptide epitope. In some embodiments, the polymorphonuclear leukocytes express at least two different CAR or TCR targeting at least two different epitopes.

In some embodiments, the polymorphonuclear leukocytes are genetically modified by mRNA transfection. In some embodiments, the polymorphonuclear leukocytes are genetically modified by viral transduction using a viral vector. In some embodiments, the viral vector is a lentiviral vector.

In some embodiments, the polymorphonuclear leukocytes are neutrophils.

In some embodiments, the cancer is lymphoma.

In another aspect provided herein is a method of treating a cancer in a subject in need thereof comprising administering to the subject a therapeutically effective amount of a polynucleotide coding for a CAR or a TCR complexed with a carrier wherein the carrier comprises one or more molecules effective to deliver the polynucleotide to the cytoplasm of a polymorphonuclear leukocyte of the subject. In some embodiments, the CAR targets 4-BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin. IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

In some embodiments, the polymorphonuclear leukocytes express a chimeric antigen receptor (CAR) targeting CD19. In some embodiments, the CD19-targeting CAR comprises the amino acid sequence of SEQ ID NO: 3. In some embodiments, the CD19-targeting CAR is encoded by the nucleotide sequence of SEQ ID NO: 1.

In some embodiments, the polymorphonuclear leukocytes express a chimeric antigen receptor (CAR) targeting a human B cell receptor (BCR). In some embodiments, the BCR-targeting CAR comprises the amino acid sequence of SEQ ID NO: 4. In some embodiments, the BCR-targeting CAR is encoded by the nucleotide sequence of SEQ ID NO: 2.

In some embodiments, the polymorphonuclear leukocytes express a chimeric antigen receptor (CAR) targeting mesothelin. In some embodiments, the mesothelin-targeting CAR comprises the amino acid sequence of SEQ ID NO: 6. In some embodiments, the mesothelin-targeting CAR is encoded by the nucleotide sequence of SEQ ID NO: 5.

In some embodiments, the CAR or the TCR targets a tumor-specific peptide epitope. In some embodiments, the CAR or the TCR targets at least two different tumor-specific peptide epitopes.

In some embodiments, the polynucleotide is mRNA.

In some embodiments, the carrier is a recombinant histone H1 molecule. In some embodiments, the recombinant histone H1 molecule is a human recombinant H1.3 molecule. In some embodiments, the human recombinant H1.3 molecule additionally comprises an N-terminal bis-methionine sequence and the N,N-bismethionylhistone H1.3 molecule comprises the amino acid sequence of SEQ ID NO: 8.

In some embodiments, the carrier is a liposome. In some embodiments, the liposome is conjugated with a targeting moiety specific for the polymorphonuclear leukocyte. In some embodiments, the targeting moiety is a CCXP1 peptide and comprises the amino acid sequence of Met-Leu-Trp-Arg-Arg-Lys-Ile-Gky-Pro-Gln-Met-Thr-Leu-Ser-Ala-Gly (SEQ ID NO: 7).

In some embodiments, the polymorphonuclear leukocytes are neutrophils.

In some embodiments, the cancer is lymphoma. In some embodiments, the cancer is pancreatic adenocarcinoma.

In another aspect provided herein is a modified polymorphonuclear leukocyte, wherein the polymorphonuclear leukocyte expresses a recombinant chimeric antigen receptor (CAR) or T cell receptor (TCR). In some embodiments, the polymorphonuclear leukocyte expresses a chimeric antigen receptor (CAR) targeting 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

In some embodiments, the polymorphonuclear leukocyte expresses a chimeric antigen receptor (CAR) targeting CD19. In some embodiments, the CD19-targeting CAR comprises the amino acid sequence of SEQ ID NO: 3. In some embodiments, the CD19-targeting CAR is encoded by the nucleotide sequence of SEQ ID NO: 1.

In some embodiments, the polymorphonuclear leukocyte expresses a chimeric antigen receptor (CAR) targeting a human B cell receptor (BCR). In some embodiments, the BCR-targeting CAR comprises the amino acid sequence of SEQ ID NO: 4. In some embodiments, the BCR-targeting CAR is encoded by the nucleotide sequence of SEQ ID NO: 2.

In some embodiments, the polymorphonuclear leukocytes express a chimeric antigen receptor (CAR) targeting mesothelin. In some embodiments, the mesothelin-targeting CAR comprises the amino acid sequence of SEQ ID NO: 6. In some embodiments, the mesothelin-targeting CAR is encoded by the nucleotide sequence of SEQ ID NO: 5.

In some embodiments, the CAR or the TCR targets a tumor-specific peptide epitope. In some embodiments, the polymorphonuclear leukocyte expresses at least two different CAR or TCR targeting at least two different epitopes.

In some embodiments, the polymorphonuclear leukocyte is a neutrophil.

In another aspect provided herein is a pharmaceutical composition comprising the modified polymorphonuclear leukocyte described herein and a pharmaceutical acceptable carrier or excipient.

### BRIEF DESCRIPTION OF THE DRAWINGS

This patent application file contains at least one drawing executed in color. Copies of this patent application with color drawing(s) will be provided by the Office upon request and payment of the necessary fee.

FIG. 1 shows a graph of data from analysis of the transduced neutrophils survival after transduction in comparison to control.

FIG. 2 shows two graphs of flow cytometry analysis of expression of the CD19-CAR by neutrophils.

FIG. 3 shows a graph of cytotoxicity data of CAR19 or mock polymorphonuclear leukocytes co-cultured with human Burkitt lymphoma cells line Raji. Cytotoxicity was determined by measuring lactate dehydrogenase release after 6 hours.

FIG. 4 is a graph showing that polymorphonuclear leukocytes modified by CAR19 effectively suppress Raji tumor growth in NOD SCID mice, in comparison with tumor-bearing NOD SCID mice treated with unmodified polymorphonuclear leukocytes.

FIGS. 5A-5C show three graphs of flow cytometry analysis of CD19 CAR expressing neutrophils (FIG. 5A), neoepitope CAR expressing neutrophils (FIG. 5B), and dual neoepitope CAR/CD19 CAR expressing neutrophils (FIG. 5C).

FIG. 6A is a graph showing that polymorphonuclear leukocytes expressing CAR19 (Group 1), neoepitope CAR (Group 2), and dual neoepitope CAR/CD19 CAR (Group 3) effectively suppress Raji tumor grow in SCID mice. FIG. 6B shows a phase contrast and fluorescence photograph of polymorphonuclear leukocytes incubated with mRNAs encoding CD19 CAR alone. FIG. 6C shows a phase contrast and fluorescence photograph of polymorphonuclear leukocytes incubated with mRNA encoding CAR targeting CD19/N,N-bismethionylhistone H 1.3 complex.

FIG. 7 is a graph showing that intravenous injection of the CAR19 mRNA/H1.3 effectively suppresses Raji tumor grow in SCID mice in comparison to control Group 2 (mRNA CAR alone) and Group 3 (H1.3 alone).

FIG. 8 has three graphs showing flow cytometry analysis of CD19 CAR expressing polymorphonuclear leukocytes of mice treated with mRNA CAR19/H1.3 (Group 1), mRNA CAR alone (Group 2) or H1.3 alone (Group 3).

FIG. 9 shows bioluminescence data of mice treated with PBS in the control group (top panel), CCXP1 targeted empty liposomes (middle panel), and CCXP1 targeted liposomes loaded with scFv-TM8-4-1BB-CD3z CAR mRNA (bottom panel).

FIG. 10 shows three graphs illustrating flow cytometry analysis of scFv-TM8-4-1BB-CD3z CAR expressing polymorphonuclear leukocytes from experimental groups.

### DETAILED DESCRIPTION

Unless defined otherwise, technical and scientific terms used herein have the same meaning as commonly understood by one of ordinary skill in the art to which this invention belongs.

The terms “a,” “an,” and “the” do not denote a limitation of quantity, but rather denote the presence of “at least one” of the referenced item.

The terms “patient”, “individual”, “subject”, “mammal”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. The exact amount required will vary from subject to subject, depending on the species, age, and general condition of the subject, the severity of the condition being treated, the particular drug or drugs employed, the mode of administration, and the like.

The terms and expressions which have been employed are used as terms of description and not of limitation, and use of such terms and expressions do not exclude any equivalents of the features shown and described or portions thereof, and various modifications are possible within the scope of the technology claimed.

In one aspect, the present invention provides a method of treating a cancer in a subject in need thereof. The method comprises administering to the subject a therapeutically effective amount of polymorphonuclear leukocytes (PMNs) genetically modified to express a recombinant chimeric antigen receptor (CAR) or T cell receptor (TCR).

CARs of the present invention typically comprise (i) an extracellular antigen-binding domain, which may comprise an antibody or an antibody fragment (e.g., a single-chain variable fragment (scFv) derived from an antigen-specific monoclonal antibody) and (ii) a lymphocyte activation domain (e.g., a lymphocyte activation domain derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, or MyD88). These two domains are connected via a transmembrane domain (e.g., a transmembrane domain derived from CD3ζ, CD28, CD4, or CD8α). Upon CAR contact with the target antigen, it signals through the lymphocyte activation domain inducing cytotoxicity and cellular activation. CARs can also contain co-stimulatory domain(s) which boost the CAR-induced immune response (e.g., CD28 or 4-1BB co-stimulatory domains). Examples of known CARs include CD19 CARs described in Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533, each of which is herein incorporated by reference in their entirety for all purposes.

Characteristics of CARs of the present invention include their ability to redirect polymorphonuclear leukocyte specificity and reactivity toward a selected target in a non-MHC-restricted manner, exploiting the antigen-binding properties of monoclonal antibodies. The non-MHC-restricted antigen recognition gives polymorphonuclear leukocytes expressing CARs the ability to recognize antigen independent of antigen processing, thus bypassing a major mechanism of tumor escape. The CAR can specifically bind to and immunologically recognize an antigen, e.g., a tumor-specific antigen, such that binding of the CAR to the antigen elicits an immune response.

An antigen can be any molecule (e.g., protein, peptide, polysaccharide, glycoprotein, glycolipid, nucleic acid, portions thereof, or combinations thereof) that is capable of being bound by an antibody or a T-cell receptor. An antigen is also able to provoke an immune response. An example of an immune response may involve, without limitation, antibody production, or the activation of specific immunologically competent cells, or both. Non-limiting examples of antigen-binding domains in CARs described herein include antibodies and antibody fragments such as e.g., monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above.

In various embodiments, the CAR targets CD19. The CAR may be encoded by a polynucleotide comprising the sequence of SEQ ID NO: 1. The CAR may be encoded by a sequence at least 85%, 86%, 87%, 88%, 89%, 90%, 91%, 92%, 93%, 94%, 95%, 96%, 97%, 98%, 99% identical to the sequence of SEQ ID NO: 1. The CAR may comprise the sequence of SEQ ID NO: 3. The CAR may comprise a sequence at least 85%, 86%, 87%, 88%, 89%, 90%, 91%, 92%, 93%, 94%, 95%, 96%, 97%, 98%, 99% identical to the sequence of SEQ ID NO: 3. The CAR can be about 50 to about 5000 amino acids long, such as 50, 70, 75, 100, 125, 150, 175, 200, 300, 400, 500, 600, 700, 800, 900, 1000 or more amino acids in length.

In various embodiments, the CAR targets a human B cell receptor. The CAR may be encoded by a polynucleotide comprising the sequence of SEQ ID NO: 2. The CAR may be encoded by a sequence at least 85%, 86%, 87%, 88%, 89%, 90%, 91%, 92%, 93%, 94%, 95%, 96%, 97%, 98%, 99% identical to the sequence of SEQ ID NO: 2. The CAR may comprise the sequence of SEQ ID NO: 4. The CAR may comprise a sequence at least 85%, 86%, 87%, 88%, 89%, 90%, 91%, 92%, 93%, 94%, 95%, 96%, 97%, 98%, 99% identical to the sequence of SEQ ID NO: 4. The CAR can be about 50 to about 5000 amino acids long, such as 50, 70, 75, 100, 125, 150, 175, 200, 300, 400, 500, 600, 700, 800, 900, 1000 or more amino acids in length.

In various embodiments, the CAR targets mesothelin. The CAR may be encoded by a polynucleotide comprising the sequence of SEQ ID NO: 5. The CAR may be encoded by a sequence at least 85%, 86%, 87%, 88%, 89%, 90%, 91%, 92%, 93%, 94%, 95%, 96%, 97%, 98%, 99% identical to the sequence of SEQ ID NO: 5. The CAR may comprise the sequence of SEQ ID NO: 6. The CAR may comprise a sequence at least 85%, 86%, 87%, 88%, 89%, 90%, 91%, 92%, 93%, 94%, 95%, 96%, 97%, 98%, 99% identical to the sequence of SEQ ID NO: 6. The CAR can be about 50 to about 5000 amino acids long, such as 50, 70, 75, 100, 125, 150, 175, 200, 300, 400, 500, 600, 700, 800, 900, 1000 or more amino acids in length.

Sequence of cDNA encoding the CD19 CAR (SEQ ID NO: 1):

ATGCTGCTGCTGGTCACTTCTCTGCTGCTGTGCGAACTGCCCCACCCCGC
CTTTCTGCTGATTCCCGACATCCAGATGACACAGACTACATCCTCCCTGT
CTGCCTCTCTGGGAGACAGAGTCACCATCAGTTGCAGGGCAAGTCAGGAC
ATTAGTAAATATTTAAATTGGTATCAGCAGAAACCAGATGGAACTGTTAA
ACTCCTGATCTACCATACATCAAGATTACACTCAGGAGTCCCATCAAGGT
TCAGTGGCAGTGGGTCTGGAACAGATTATTCTCTCACCATTAGCAACCTG
GAGCAAGAAGATATTGCCACTTACTTTTGCCAACAGGGTAATACGCTTCC
GTACACGTTCGGAGGGGGGACCAAGCTGGAGATCACAGGTGGCGGTGGCT
CGGGCGGTGGTGGGTCGGGTGGCGGCGGATCTGAGGTGAAACTGCAGGAG
TCAGGACCTGGCCTGGTGGCGCCCTCACAGAGCCTGTCCGTCACATGCAC
TGTCTCAGGGGTCTCATTACCCGACTATGGTGTAAGCTGGATTCGCCAGC
CTCCACGAAAGGGTCTGGAGTGGCTGGGAGTAATATGGGGTAGTGAAACC
ACATACTATAATTCAGCTCTCAAATCCAGACTGACCATCATCAAGGACAA
CTCCAAGAGCCAAGTTTTCTTAAAAATGAACAGTCTGCAAACTGATGACA
CAGCCATTTACTACTGTGCCAAACATTATTACTACGGTGGTAGCTATGCT
ATGGACTACTGGGGCCAAGGAACCTCAGTCACCGTCTCCTCAGAACAGAA
ACTGATTTCCGAGGAAGATCTGTTCGTCCCCGTGTTCCTGCCTGCCAAGC
CAACAACTACCCCTGCTCCACGACCACCTACTCCAGCACCTACCATCGCA
AGTCAGCCCCTGTCACTGCGACCTGAGGCTTGCCGGCCAGCAGCTGGAGG
AGCAGTGCACACCCGAGGCCTGGACTTCGCATGCGATATCTACATTTGGG
CACCACTGGCTGGAACCTGTGGGGTCCTGCTGCTGAGCCTGGTCATCACC
CTGTATTGTAACCACAGAAATAGGAGCAAACGCTCCCGACTGCTGCATTC
CGACTACATGAACATGACACCTCGGAGACCAGGCCCCACTAGAAAGCATT
ACCAGCCATATGCCCCACCCAGGGATTTCGCAGCCTATCGGAGCCGGTTC
AGCGTCGTGAAAAGGGGGCGCAAGAAACTGCTGTACATCTTCAAGCAGCC
TTTTATGCGCCCAGTGCAGACAACTCAGGAGGAAGACGGATGCTCTTGTC
GGTTCCCAGAGGAGGAGGAAGGAGGCTGCGAGCTGAGAGTGAAGTTCAGC
CGGAGCGCCGATGCACCAGCATATCAGCAGGGACAGAATCAGCTGTACAA
CGAGCTGAATCTGGGCAGGCGCGAGGAATATGACGTGCTGGATAAGCGAC
GAGGACGGGACCCCGAAATGGGAGGAAAACCCAGAAGGAAGAACCCTCAG
GAGGGGCTGTATAATGAACTGCAGAAAGACAAGATGGCTGAGGCATACAG
CGAAATTGGAATGAAAGGAGAGCGCCGACGGGGGAAGGGACACGATGGGC
TGTACCAGGGACTGTCAACCGCCACTAAAGATACCTACGACGCACTGCAC
ATGCAGGCTCTGCCCCCAAGA

Sequence of cDNA encoding the neoepitope CAR targeting clonal human B cell receptor transgenically expressed in Raji-FL1 lymphoma cell line (SEQ ID NO: 2):

ATUTACAGGATGCAACTCCTGTCTTGCATTGCACTAAGTCTTGCACTTGT
CACGAATTCGGCTTGTATTCTTGATTTGCCGAAGTTTTGCGGTGGAGGTT
CGGCTAGCGAGCCCAAATCTTGTGACAAAACTCACACATGCCCACCGTGC
CCAGCACCTGAACTCCTGGGGGGACCGTCAGTCTTCCTCTTCCCCCCAAA
ACCCAAGGACACCCTCATGATCTCCCGGACCCCTGAGGTCACATGCGTGG
TGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACC
ACAGGTGTACACCCTGCCCCCA7TCCCGGGATGAGCTGACCAAGAACCAG
GTCAGCCTGACCTGCCTGGTCAAAGGCTTCTATCCCAGCGACATCGCCGT
GGAGTGGGAGAGCAATGGGCAGCCGGAGAACAACTACAAGACCACGCCTC
CCGTGCTGGACTCCGACGGCTTCCTTCTTCCTCTACAGCAAGCTCACCGT
GGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCG
GGTAAAGGCTCCACCAGCGGCAGCGGCAAGCCAGGCTCCGGCGAAGGCAG
CACCAAAGGCTTCTGGGTCCTGGTCGTGGTGGGGGGGGTGTTGGCCTGTT
ATAGCCTGCTGGTGACTGTCGCCTTCATTATATTTTGGGTGAGATCCAAA
AGATCCCGGCTGCTCCATTCTGACTACATGAACATGACTCCAAGGCGACC
AGGACCTACTAGAAAGCATTATCAGCCGTACGCTCCACCCCGGGATTTTG
CGGCTTATCGAAGTAGGGACCAGAGATTGCCCCCTGACGCCCACAAGCCT
CCCGGGGGGGGAAGCTTCCGCACCCCCATCCAGGAAGAACAAGCAGATGC
ACATAGTACTCTGGCCAAAATAAGAGTGAAGTTTTCTAGAAGCGCAGACG
CACCAGCTTACCAGCAGGGCCAAAATCAGCTGTACAACGAGCTGAACCTG
GGCCGGAGGGAGGAATACGATGTGCTGGATAAGCGCCGGGGCCGCGATCC
CGAAATGGGCGGCAAGCCACGGCGAAAGAATCCTCAGGAGGGACTGTACA
ACGAACTCCAAAAGGACAAGATGGCGGAGGCATACTCCGAGATCGGCATG
AAGGGGGAAAGACGGCGGGGCAAGGGGCATGACGGTCTGTATCAAGGGTT
GAGTACCGCCACAAAGGATACCTACGACGCCCTTCACATGCAGGCCCTGC
CCCCTCGCTAGTAA

Sequence of CD19 CAR (SEQ ID NO: 3) encoded by the polynucleotide of SEQ ID NO: 1:

MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGGGGSGGGGSGGGGSEVKLQE
SGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYA
MDYWGQGTSVTVSSEQKLISEEDLFVPVFLPAKPTTTPAPRPPTPAPTIA
SQPLSLRPEACRPAAGGAVHTRGLDFACDIYIWAPLAGTCGVLLLSLVIT
LYCNHRNRSKRSRLLHSDYMNMTPRRPGPTRKHYQPYAPPRDFAAYRSRF
SVVKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALH
MQALPPR

Sequence of neoepitope CAR targeting clonal human B cell receptor transgenically expressed in Raji-FL1 lymphoma cell line (SEQ ID NO: 4) encoded by the polynucleotide of SEQ ID NO: 2:

MYRMQLLSCIALSLALVTNSACILDLPKFCGGGSASEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGEYPSDIAV
EWESNGQPENNYKTTPPVLDSDGSFFLYSKLTVDKSRWQQGNVFScSVMH
EALHNHYTQKSLSLSPGKGSTSGSGKPGSGEGSTKGFWVLVVVGGVLACY
SLLVTVAFIIFWVRSKRSRLLHSDYMNMTPRRPGPTRKHYQPYAPPRDFA
AYRSRDQRLPPDAHKPPGGGSFRTPIQEEQADAHSTLAKIRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR

Sequence of cDNA encoding the mesothelin CAR (SEQ ID NO: 5):

ATGCAGGTACAACTGCAGCAGTCTGGGCCTGAGCTGGAGAAGCCTGGCGC
TTCAGTGAAGATATCCTGCAAGGCTTCTGGTTACTCATTCACTGGCTACA
CCATGAACTGGGTGAAGCAGAGCCATGGAAAGAGCCTTGAGTGGATTGGA
CTTATTACTCCTTACAATGGTGCTTCTAGCTACAACCAGAAGTTCAGGGG
CAAGGCCACATTAACTGTAGACAAGTCATCCAGCACAGCCTACATGGACC
TCCTCAGTCTGACATCTGAAGACTCTGCAGTCTATTTCTGTGCAAGGGGG
GGTTACGACGGGAGGGGTTTTGACTACTGGGGCCAAGGGACCACGGTCAC
CGTCTCCTCAGGTGTAGGCGGTTCAGGCGGCGGTGGCTCTGGCGGTGGCG
GATCGGACATCGAGCTCACTCAGTCTCCAGCAATCATGTCTGCATCTCCA
GGGGAGAAGGTCACCATGACCTGCAGTGCCAGCTCAAGTGTAAGTTACAT
GCACTGGTACCAGCAGAAGTCAGGCACCTCCCCCAAAAGATGGATTTATG
ACACATCCAAACTGGCTTCTGGAGTCCCAGGTCGCTTCAGTGGCAGTGGG
TCTGGAAACTCTTACTCTCTCACAATCAGCAGCGTGGAGGCTGAAGATGA
TGCAACTTATTACTGCCAGCAGTGGAGTGGTTACCCTCTCACGTTCGGTG
CTGGGACAAAGTTGGAAATAAAATCCGGAACCACGACGCCAGCGCCGCGA
CCACCAACACCGGCGCCCACCATCGCGTCGCAGCCCCTGTCCCTGCGCCC
AGAGGCGTGCCGGCCAGCGGCGGGGGGCGCAGTGCACACGAGGGGGCTGG
ACTTCGCCTGTGATATCTACATCTGGGCGCCCTTGGCCGGGACTTGTGGG
GTCCTTCTCCTGTCACTGGTTATCACCCTTTACTGCAAACGGGGCAGAAA
GAAACTCCTGTATATATTCAAACAACCATTTATGAGACCAGTACAAACTA
CTCAAGAGGAAGATGGCTGTAGCTGCCGATTTCCAGAAGAAGAAGAAGGA
GGATGTGAACTGAGAGTGAAGTTCAGCAGGAGCGCAGACGCCCCCGCGTA
CAAGCAGGGCCAGAACCAGCTCTATAACGAGCTCAATCTAGGACGAAGAG
AGGAGTACGATGTTTTGGACAAGAGACGTGGCCGGGACCCTGAGATGGGG
GGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCC
ACCAAGGACACCTACGACGCCCTTCACATGCAGGCCCTGCCCCCTCGCTA
A

Sequence of mesothelin CAR (SEQ ID NO: 6) encoded by the polynucleotide of SEQ ID NO: 5:

MQVQLQQSGPELEKPGASVKISCKASGYSFTGYTMNWVKQSHGKSLEWIG
LITPYNGASSYNQKFRGKATLTVDKSSSTAYMDLLSLTSEDSAVYFCARG
GYDGRGFDYWGQGTTVTVSSGVGGSGGGGSGGGGSDIELTQSPAIMSASP
GEKVTMTCSASSSVSYMEIWYQQKSGTSPKRWIYDTSKLASGVPGRFSGS
GSGNSYSLTISSVEAEDDATYYCQQWSGYPLTFGAGTKLEIKSGTTTPAP
RPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEE
GGCELRVKFSRSADAPAYKQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR

In various embodiments, the CAR targets 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

In various embodiments, the polymorphonuclear leukocytes express a CAR or a TCR targeting a tumor-specific peptide epitope. Exemplary tumor-specific peptide epitopes comprise a contiguous 8-11 amino acid sequence from one or more of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin. The epitope may be a neoepitope.

In various embodiments, the polymorphonuclear leukocytes express at least two different CAR(s) or TCR(s) targeting at least two different epitopes. In various embodiments, the polymorphonuclear leukocytes express a CAR targeting an epitope, and a TCR targeting an epitope that is different from the epitope targeted by the CAR.

The term “cancer” refers to the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, lymphoma, carcinoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), pancreatic adenocarcinoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. In some embodiments, the cancer is lymphoma. In some embodiments, the cancer is pancreatic adenocarcinoma.

The term “transfection” refers to the introduction of a foreign (i.e., extrinsic or extracellular) nucleic acid (e.g., mRNA) into a cell. The terms “genetically modified” or “genetic modification” refer to the introduction of a foreign (i.e., extrinsic or extracellular) gene, DNA or RNA sequence to a cell, so that the cell will express the introduced gene or sequence to produce a desired substance, typically a protein or enzyme coded by the introduced gene or sequence. The introduced gene or sequence may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function.

In various embodiments, the polymorphonuclear leukocytes are genetically modified by mRNA or cDNA transfection, wherein the mRNA or cDNA encodes the CAR or the TCR. The construct used for transfection may be a plasmid or a viral vector.

Various transfection methods may be used to deliver a polynucleotide (e.g., a mRNA or cDNA) into a cell, including but not limited to histone-mediated transfection and liposome-mediated transfection. Histones are cationic proteins which naturally compact DNA. They are responsible in vivo for compacting DNA is not transcribed, and the DNA of certain viruses. Non-limiting examples of histone molecules that may be used include linker histones such as histone H1 (e.g., H1.3 histone), H1 C-terminal peptide, Histone H1.4F peptide, H1 C-Terminal peptide, and Galactosylated H1; core histones such as H2A, H2B, H3, H4; and other histone-like protein such as TmHU, HPhA, thermophilic histone, and HU from Bifidobacterium longuem (see Han et al., Biotechnol. Adv. 2019; 37(1):132-144, which is incorporated herein by reference in its entirety). Other proteins that may also be used in facilitate transfection of a foreign nucleic acid (e.g., mRNA) include protamines and nucleolins (see e.g., U.S. Pat. No. 6,200,956, which is incorporated herein by reference in its entirety). In liposome-mediated transfection, polynucleotides may be entrapped in a lipid complex and suspended in an excess of aqueous solution for delivery (See e.g., Ghosh and Bachhawat, (1991) In: Liver Diseases, Targeted Diagnosis and Therapy Using Specific Receptors and Ligands. pp. 87-104, which is incorporated herein by reference in its entirety). The liposome may be additionally conjugated with a targeting moiety (e.g., a peptide) that is capable of directing the liposome and the polynucleotide to a target tissue or cell type. A non-limiting example of a targeting peptide for polymorphonuclear leukocytes is CCXP1 (Met-Leu-Trp-Arg-Arg-Lys-Ile-Gky-Pro-Gln-Met-Thr-Leu-Ser-Ala-Gly; SEQ ID NO: 7). CCXP1 peptide has targeting capacity to formyl peptide receptor 1 (FPR1) which is expressed on polymorphonuclear leukocytes.

Additional transfection methods contemplated by the present disclosure include using a polynucleotide complexed with Lipofectamine, or Superfect; DEAE-dextran (e.g., a polynucleotide is delivered into a cell using DEAE-dextran followed by polyethylene glycol. See e.g., Gopal, T. V., Mol. Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and/or polypeptide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In some embodiments, the polynucleotide encoding a CAR or a TCR is introduced to the polymorphonuclear leukocyte via a viral vector. In some embodiments, the viral vector can be, but is not limited to, adenoviral vector, an adeno-associated virus vector, an alphaviral vector, a herpes virus vector, a lentiviral vector, a retroviral vector, and vaccinia virus vector. In some embodiments, the viral vector is a lentiviral vector.

Polymorphonuclear leukocytes are a family of white blood cells including immune cells such as neutrophils, eosinophils, and basophils. In various embodiments, the polymorphonuclear leukocytes are neutrophils. The neutrophils may be effective to phagocytose a cell, e.g., a tumor cell, expressing the target of the CAR or the TCR. The neutrophils may also generate neutrophil extracellular traps (NETs). In some embodiments, the polymorphonuclear leukocytes are eosinophils. In some embodiments, the polymorphonuclear leukocytes are basophils.

The polymorphonuclear leukocytes may be autologous/autogenetic (“self”) or non-autologous (“non-self,” e.g., allogeneic, syngeneic or xenogeneic). In some embodiments, the polymorphonuclear leukocytes are obtained from a mammalian subject. In some embodiments, the polymorphonuclear leukocytes are obtained from a human subject.

Polymorphonuclear leukocytes may be isolated using methods known in the art. For example, human polymorphonuclear leukocytes can be obtained from venous blood using an anti-coagulant (e.g., 10% heparin) followed by centrifugation of the blood on Ficoll-Hypaque gradients. Contaminating erythrocytes can be removed by hypotonic lysis. The remaining cell population would consist of 95 to 98% polymorphonuclear leukocytes, and these cells can be suspended in Hank's balanced salt solution pH 7.4 at a desired concentration (see U.S. Pat. No. 5,166,133, which is hereby incorporated by reference in its entirety).

In another aspect, the invention provides a method of treating a cancer in a subject in need thereof. A therapeutically effective amount of polynucleotide coding for a CAR, or a TCR complexed with a carrier, is administered to the subject. The carrier comprises one or more molecules effective to deliver a polynucleotide to the cytoplasm of a polymorphonuclear leukocyte of the subject.

In various embodiments, the CAR targets 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, 1GF, human scatter factor receptor kinase, mesothelin. IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

In various embodiments, the CAR or the TCR targets a tumor-specific peptide epitope. In various embodiments, CAR or the TCR targets at least two different epitopes. In various embodiments, the polymorphonuclear leukocytes express a CAR targeting an epitope, and a TCR targeting an epitope that is different from the epitope targeted by the CAR.

The polynucleotide may be an mRNA. The carrier may be a recombinant histone H1 molecule, for example but not limited to a human recombinant H1.3 molecule. The human recombinant H1.3 molecule additionally comprises an N-terminal bis-methionine sequence.

The N,N-bismethionylhistone H1.3 (H1.3 histone) recombinant protein is a single stranded sequence with 222 amino acid residues. The molecular weight is 22.5 kDa. The amino acid sequence (SEQ ID NO: 8) of N,N-bismethionylhistone H1.3 histone:

MNISETAPLAPTIPAPAEKTPVKKKAKKAGATAGKRKASGPPVSELITKA
VAASKERSGVSLAALKKALAAAGYDVEKNNSRIKLGLKSLVSKGTLVQTK
GTGASGSFKLNKKAASGEGKPKAKKAGAAKPRKPAGAAKKPKKVAGAATP
KKSIKKTPKKVKKPATAAGTKKVAKSAKKVKTPQPKKAAKSPAKAKAPKP
KAAKPKSGKPKVTKAKKAAPKKK

In various embodiments, the polymorphonuclear leukocytes are neutrophils. In some embodiments, the polymorphonuclear leukocytes are eosinophils. In some embodiments, the polymorphonuclear leukocytes are basophils.

In one aspect, the invention provides a modified polymorphonuclear leukocyte wherein the polymorphonuclear leukocyte is genetically modified to express a CAR or TCR.

In various embodiments, the polymorphonuclear leukocytes express a CAR targeting 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

In various embodiments, the CAR or the TCR targets a tumor-specific peptide epitope. In various embodiments, CAR or the TCR targets at least two different epitopes. In various embodiments, the polymorphonuclear leukocytes express a CAR targeting an epitope, and a TCR targeting an epitope that is different from the epitope targeted by the CAR.

In some embodiments, the polymorphonuclear leukocyte is genetically modified by mRNA transfection. In some embodiments, the polymorphonuclear leukocytes are neutrophils.

In one aspect is provided a pharmaceutical composition comprising the modified polymorphonuclear leukocytes described herein. The pharmaceutical composition may comprise one or more pharmaceutically acceptable carriers.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

Examples of pharmaceutically acceptable carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified polymorphonuclear leukocytes disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified polymorphonuclear leukocytes disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Development and Production of CD19-CAR Lentivirus

The nucleotide sequence coding for chimeric antigen receptor specific to CD19 antigen (CAR19) was synthesized (Genecust, France) and cloned to the pLV2 lentiviral plasmid vector.

To produce lentiviruses, 293T packaging cells were transfected with the pLV2-CAR19 plasmid vector. The packaging plasmids were psPAX2 and pMD2.G (Invitrogen, USA). The day before transfection, 293T cells were plated in a 10 cm dish cultured in DMEM (Invitrogen, USA) with 10% FBS (Hyclone, USA). When cell density reached about 60% to about 80%, the cells were transfected with the above plasmids using polyethylenimine (PEI, Polysciences, USA). After the transfected cells incubated in a 37° C., 5% CO₂ incubator for 12-16 hours, the culture medium was replaced with 5-6 ml fresh DMEM with 10% FBS. The supernatants containing viruses were harvested at 24 hours and 48 hours and concentrated by ultracentrifugation for 90 minutes at 50,000 g, 4° C., then stored at −80° C. until use.

### Example 2: Transduction of Polymorphonuclear Leukocytes with Lentiviral Vector Coding for CD19 Targeting CAR

Human polymorphonuclear leukocytes were isolated from fresh donor blood using EasySep™ Direct Human Neutrophil Isolation kit (Stem Cell Technologies Inc, Vancouver, Canada) according to the manufacturer protocol. Processing of 1 ml of blood containing approximately 5×10⁶ human white blood cells usually yield up to 2×10⁶ 95% pure human polymorphonuclear leukocytes. Purified cells were resuspended in 500 mkl of PBS containing 2% fetal bovine serum (FBS) and 1 mM EDTA. The purity (percent of CD66b⁺CD16⁺ cells) was assessed with flow cytometry analysis with fluorochrome conjugated anti-human CD66b and CD16 antibodies from same manufacturer.

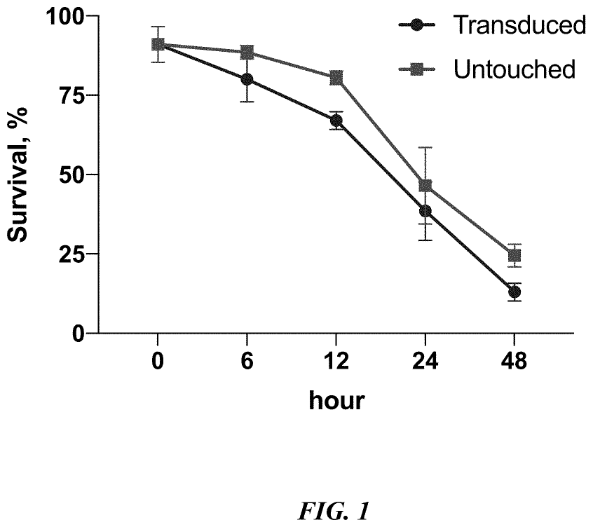

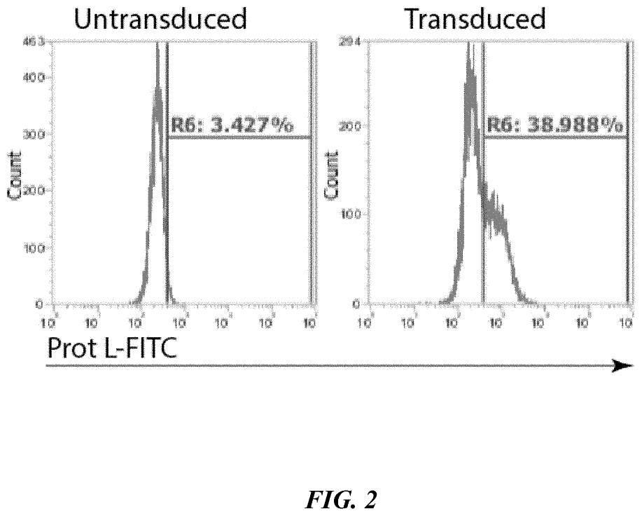

Polymorphonuclear leukocytes were spinoculated with CD19-CAR lentiviruses at 1200 g for 1 hour, and then stimulated for a further 4 hours with 50 U/ml of granulocyte-macrophage colony-stimulating factor (Neostim, Xiamen Amoytop Biotech Co., Xiamen, China). Cell survival was determined by Trypan blue assay. The data are shown in FIG. 1. Expression of CD19-CAR molecules were detected flow cytometry using biotinylated protein L (Thermo Fisher Scientific) and streptavidin conjugated with FITC (Thermo Fisher Scientific). The data are shown in FIG. 2.

The data shown in FIGS. 1 and 2 clearly confirm that polymorphonuclear leukocytes can be safely genetically reprogrammed to express chimeric antigen receptor.

### Example 3: Polymorphonuclear Leukocytes Transduced by Lentiviral Vector Coding for CD19 CAR Kill Lymphoma Cells In Vitro

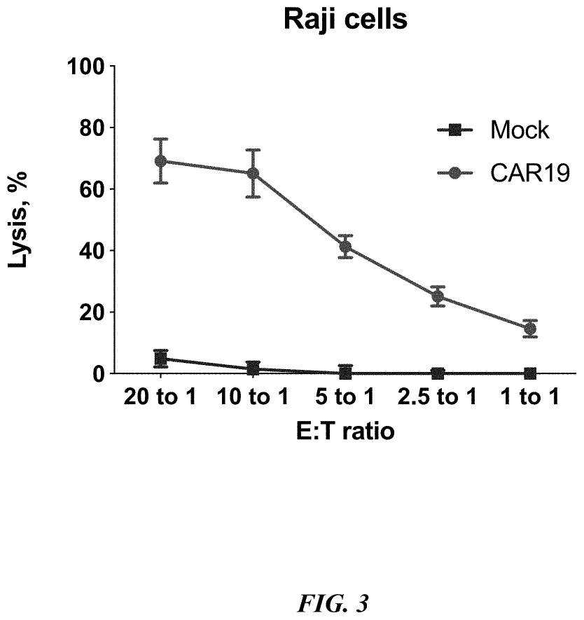

The cytotoxicity of engineered polymorphonuclear leukocytes were evaluated in a standard LDH release assay (CytoTox 96 Non-Radioactive Cytotoxicity Assay, Promega) following the manufacturer's recommendations. Polymorphonuclear leukocytes transduced by with lentiviral vector coding for CD19 CAR or untransduced control polymorphonuclear leukocytes were coincubated at different effector/target (E:T) ratio for 6 hours together with human Burkitt lymphoma cells line (Raji). Cytotoxicity was determined by measuring lactate dehydrogenase release after 6 hours. The data are shown in FIG. 3. The data clearly show that genetic reprogramming of polymorphonuclear leukocytes to express chimeric antigen receptor significantly increase their ability to kill cancer cells harboring target for the same chimeric antigen receptor.

### Example 4: Treatment of Lymphoma in Mice by Polymorphonuclear Leukocytes Transduced with Lentiviral Vector Coding for CD19 CAR

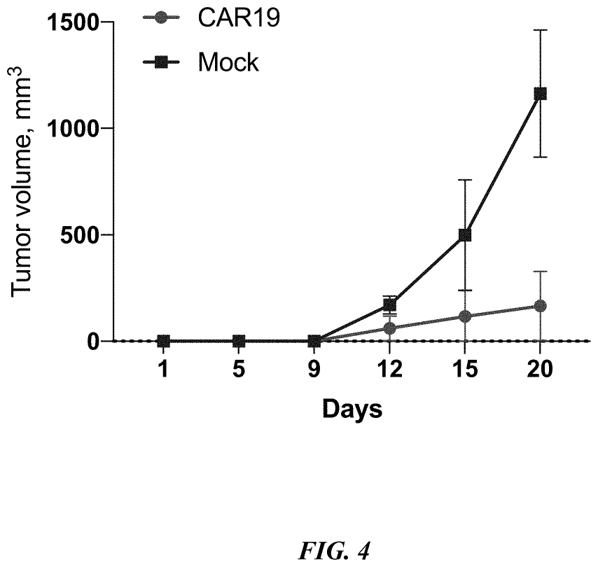

Six- to eight-week-old female NOD SCID (CB17-Prkdc^{scid}/NcrCrl) mice with an average weight of 16 to 20 g were used. Tumors were engrafted by inoculating 5×10⁶ Raji cells in 200 μl of 0.9% saline solution subcutaneously into the left side of mice. Once tumors had reached a palpable volume of at least 50 mm³, mice were randomly assigned to experimental or control groups. Tumor-bearing mice were injected intravenously with 3×10⁶ polymorphonuclear leukocytes transduced with lentiviral vector coding for CD19 CAR or untransduced control polymorphonuclear leukocytes on day 10 after tumor inoculation. Tumor volume was measured with calipers and estimated using the ellipsoidal formula. Animals were euthanized on day 20. The data are shown in FIG. 4. The data clearly show that polymorphonuclear leukocytes genetically reprogrammed to express CD19 CAR, but not negative control Mock polymorphonuclear leukocytes, provide meaningful suppression of tumor growths.

### Example 5: Establishment of Raji-FL1 Lymphoma Cell Line

Lymph node biopsies from patient with Follicular lymphoma (FL) was used to determine the nucleotide sequence of the B cell receptors (BCRs) from malignant cells. The central part of the tumor biopsy was taken in order to reduce the abundance of BCR genes from non-malignant cells. Total mRNA was used as a template in a reverse transcription reaction with subsequent PCR amplification of Ig V genes. Up to 95% percent of analyzed sequences were identical due to the clonal nature of lymphomas. Identified variable fragments of the follicular lymphoma BCRs were cloned as a scFv into the lentiviral vector pLV2-Fc-MTA coding for a membrane-anchored human antibody Fc fragment. Thus, the ScFv fused with constant domain of antibody (Fc) is linked via a flexible linker to a membrane-spanning domain of the platelet-derived growth factor receptor (PDGFR) such that the antibody molecules are integrated as dimers into the plasma membrane with their binding sites facing the solvent.

To produce lentiviruses, 293T packaging cells were transfected with pLV2-scFv-Fc-MTA plasmid vector. The packaging plasmids were psPAX2 and pMD2.G (Invitrogen, USA). The day before transfection, 293T cells were plated in a 10 cm dish cultured in DMEM (Invitrogen, USA) with 10% FBS (Hyclone, USA). When cell density reached 60%-80%, the cells were transfected with the above plasmids using polyethylenimine (PEI, Polysciences, USA). After the transfected cells incubated in 37° C., 5% CO₂ incubator for 12-16 hours, the culture medium was replaced with 5-6 ml fresh DMEM with 10% FBS. The supernatants containing viruses were harvested at 24 hours and 48 hours, concentrated by ultracentrifugation for 90 minutes at 50,000 g, 4° C., and then stored at −80° C. until use. Raji cells were transduced with collected viruses. Transduced Raji-FL were analyzed by FACS in order to select the cells carrying the follicular lymphoma BCR.

Polymorphonuclear leukocytes were transfected by mRNAs using the human monocyte nucleofector kit and the Amaxa nucleofector II device. After isolation, 5×10⁶ polymorphonuclear leukocytes were resuspended in 100 μl complete nucleofector solution containing 50 μg RNA and then transferred to a nucleoporation cuvette. Electroporation was performed using the Y001 program in the nucleofector II device. Cells were then recovered and left for 5 min in 2 ml human monocyte nucleofector medium supplemented with 2 mM glutamine and 10% FBS.

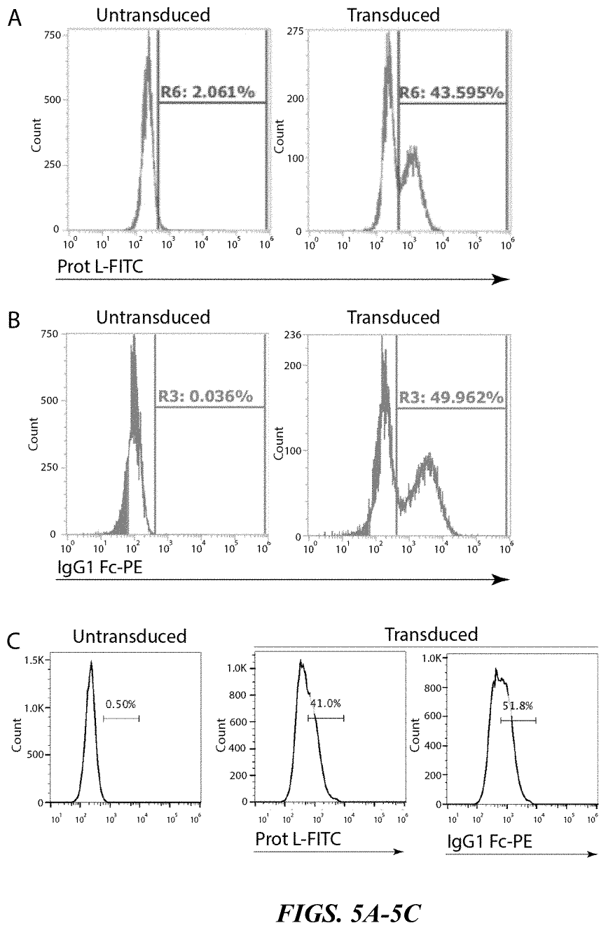

Expression of CD19-CAR molecules was detected with flow cytometry using biotinylated protein L (Thermo Fisher Scientific) and streptavidin conjugated with FITC (Thermo Fisher Scientific). The data are shown in FIG. 5A.

Expression of neoepitope CAR molecules was detected with flow cytometry using fluorescent antibodies labeled by PE specific to the Fc of the IgG1 (Southern Biotech). The data are shown in FIGS. 5B and 5C.

Six- to eight-week-old female NOD SCID (CB17-Prkdc^{scid}/NcrCrl) mice with an average weight of 16 to 20 g were used. Tumors were engrafted by inoculating 5×10⁶ Raji-FL1 cells in 200 μl of 0.9% saline solution subcutaneously into the left side of mice. Once tumors had reached a palpable volume of at least 50 mm³, mice were randomly assigned to experimental or control groups with 6 mice per group. At days 10 and 15, tumor-bearing mice were injected intravenously with 3×10⁶ polymorphonuclear leukocytes transduced with mRNA encoding CAR targeting neoepitope (Group 1; 5 mice), polymorphonuclear leukocytes transduced by mRNA encoding CAR targeting CD19 (Group 2; 5 mice) and polymorphonuclear leukocytes transduced by both mRNA encoding CAR targeting neoepitope and mRNA encoding CAR targeting CD19 (Group 3; 5 mice). Tumor volume was measured with calipers and estimated using the ellipsoidal formula. Animals were euthanized on day 20.

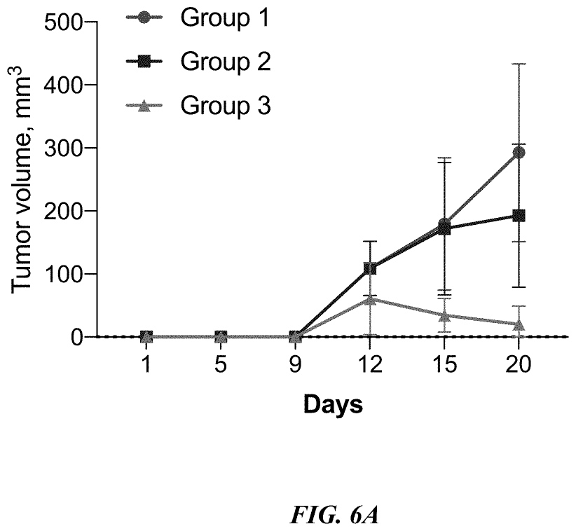

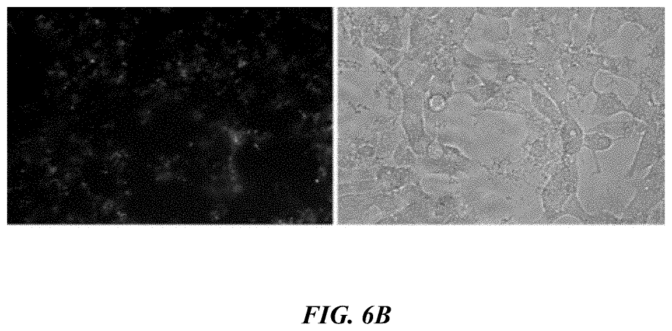

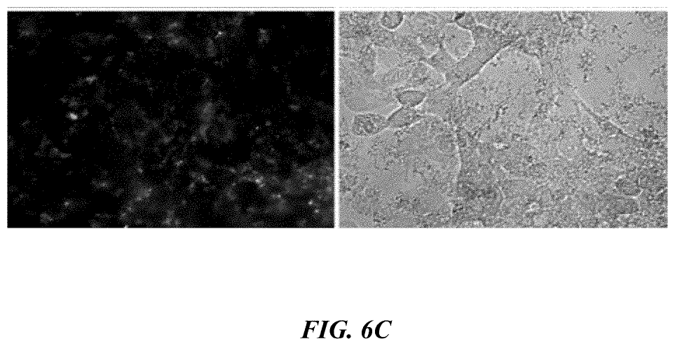

The data are shown in FIG. 6A. The data clearly show that polymorphonuclear leukocytes genetically reprogrammed by mRNA to express a CD19 CAR or CAR targeting neoepitope provide meaningful suppression of tumor growths. Surprisingly reprogramming of polymorphonuclear leukocytes to simultaneously express two different CARs provide superior efficacy for such dual reprogrammed polymorphonuclear leukocytes.

### Example 6: Transfectionally Active Complexes of mRNA Encoding CAR and Human Recombinant Histone H1.3

The predominant mechanisms through which polymorphonuclear leukocytes sense inflammatory stimuli and surrounding environments are a plethora of cell surface receptors principally within the G protein coupled receptor (GPCR) family. The first GPCR to be described on the human polymorphonuclear leukocytes was formyl peptide receptor 1 (FPR1) which, when activated, triggers a wide variety of functions, including chemotaxis, degranulation, ROS production, and phagocytosis. Constitutively expressed on the surface of quiescent polymorphonuclear leukocytes, FPR1 receptor expression is rapidly up-regulated in response to a wide number of inflammatory stimuli. The N-formylated version of any peptide containing a methionine residue at the 5′ terminus is at least 100-fold more potent than the identical nonformylated peptide. However, if the peptide contains five or more amino acids, the nonformylated moieties can also bind and activate FPR1.

The N,N-bismethionylhistone H 1.3 (H1.3 histone) recombinant protein is a single stranded sequence with 222 amino acid residues, and a molecular weight of 22.5 kDa.

Complexes between Met-Histone H1 and fluorescein-labeled mRNAs coding CD19 CAR were formed in TRIS based buffer prepared from RNase-free water suitable for storage and dilution of RNA. Ratios between mRNA and Met-Histone H1 were in the range of 1/0.2-1/10 w/w. Transfection was performed by adjustment of calcium concentration to about 4 mM and incubation of purified polymorphonuclear leukocytes with the transfection complexes for 5 h, followed by examination under the microscope for the presence of transfection complexes in the cells.

Images of phase contrast and fluorescence are made at the same focal plane. FIG. 6B shows a phase contrast and fluorescence photo of polymorphonuclear leukocytes incubated with mRNAs coding CD19 CAR alone. FIG. 6C shows a phase contrast and fluorescence photo of polymorphonuclear leukocytes incubated with mRNA encoding CAR targeting CD19/N,N-bismethionylhistone H 1.3 complex. The microscopy analysis shows that complexes between mRNAs encoding CD19 CAR/Met-Histone H1 formed in the range between 0.2-5.0 w/w mRNA/Met-Histone H1 are transfectionally active and accumulate within polymorphonuclear leukocytes; while free mRNAs encoding CD19 CAR do not enter polymorphonuclear leukocytes.

### Example 7: Intravenous Injection of Transfectionally Active Complexes of mRNA Encoding CAR and Histone H1 Leads to Transfection of Polymorphonuclear Leukocytes In Vivo

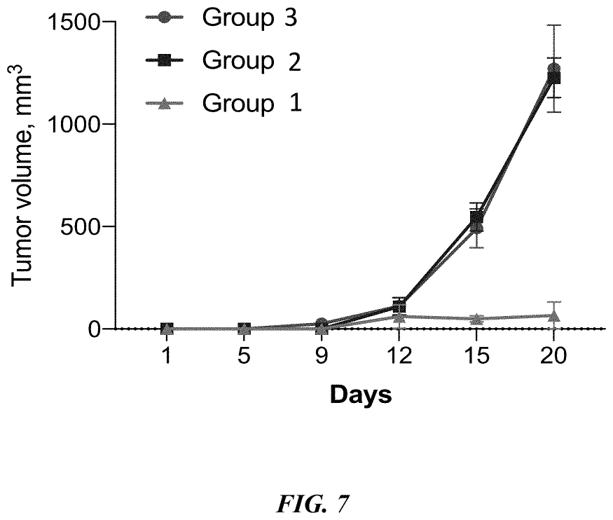

Lymphoma in mice was treated by intravenous injection of transfectionally active complexes of mRNA encoding CAR and histone H1. Six- to eight-week-old female NOD SCID (CB17-Prkdc^{scid}/NcrCrl) mice with an average weight of 16 to 20 g were used. Tumors were engrafted by inoculating 5×10⁶ Raji cells in 200 μl of 0.9% saline solution subcutaneously into the left side of mice with a caliper. Once tumors had reached an estimated volume of at least 50 mm³, mice were randomly assigned to four groups with 6 mice per group. At days 10 and 15 tumor-bearing mice were injected intravenously with equimolar doses (of approximately 100 μg) of mRNA encoding CAR targeting CD19/N,N-bismethionylhistone H 1.3 complex at 1/1 w/w mRNA/Met-Histone H1 ratio (Group 1); 100 μg of mRNA encoding CAR targeting CD19 alone (Group 2); and 100 μg of N,N-bismethionylhistone alone (Group 3). Animals were euthanized at day 20. Tumor volume was measured with calipers and estimated using the ellipsoidal formula. The data are shown in FIG. 7, and clearly indicate that intravenous injection of the CAR19 mRNA/H1.3 effectively suppresses Raji tumor growth in SCID mice in comparison with control Group 2 (mRNA CAR alone) and Group 3 (H1.3 alone).

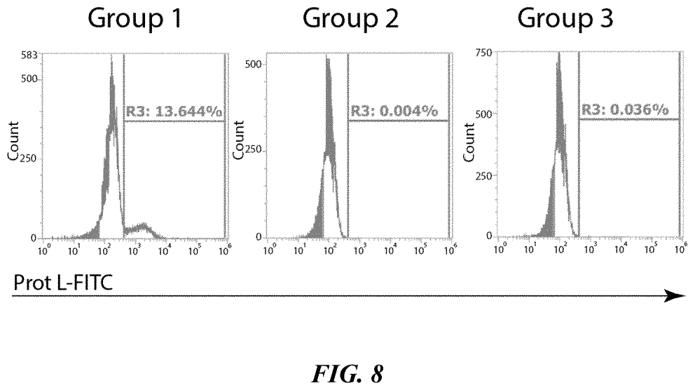

Expression of CD19-CAR molecules polymorphonuclear leukocytes was detected with flow cytometry using biotinylated protein L (Thermo Fisher Scientific) and streptavidin conjugated with FITC (Thermo Fisher Scientific). The data are shown in FIG. 8, and clearly indicate that treatment of mice with mRNA encoding CAR targeting CD19/N,N-bismethionylhistone H1.3 complex leads to expression of CD19 CAR on polymorphonuclear leukocytes and meaningful suppression of tumor growth.

### Example 8: Treatment of Pancreatic Adenocarcinoma in Mice by Intravenous Injection mRNA Encoding Mesothelin CAR Encapsulated in Liposomes Targeted to Formyl Peptide Receptor

Translationally active mRNAs encoding a mesothelin CAR scFv-TM8-4-1BB-CD3z (SEQ ID NO: 5) were produced by Promab Biotechnologies Inc. The targeting peptide CCXP1 (Met-Leu-Trp-Arg-Arg-Lys-Ile-Gky-Pro-Gln-Met-Thr-Leu-Ser-Ala-Gly) with known FPR1 targeting capacity was synthesized and purified by reverse phase high performance liquid chromatography to >95% purity at PAO Pharmsynthez (St. Petersburg, Russia). CCXP1-conjugated liposomes containing mesothelin scFv-TM8-4-1BB-CD3z CAR were prepared by coupling of CCXP1 to N-hydroxysuccinimido-carboxyl-polyethylene glycol (MW, 3400)-derived distearoylphosphatidyl ethanolamine at a 1:1.5 molar ratio. scFv-TM8-4-1BB-CD3z CAR mRNA was encapsulated in liposomes through a remote loading method at a concentration of 1 mg of mRNA per 10 μmol phospholipids.

Female BALB/c nude mice aged 6-8 weeks, and weighing approximately 19-22 grams, were anesthetized with 75 mg/kg ketamine plus 3.75 mg/kg xylazine. A left lateral abdominal incision was made, the peritoneum was opened, and the part of the pancreas near the portal area of the spleen was well exposed. BxPC-3-Luc cells (5×10⁶) in 100 μl DPBS were injected into the pancreas of mice (day 0). The abdominal wall and skin were then closed with surgical suture. On day 12, mice with established orthotopic BxPC-3-Luc tumors were randomized (8 mice/group) into 3 groups using randomized block design based upon tumor bioluminescence. Mice from control group 3 were treated with PBS by IV injection at day 15 and day 21. Mice from group 1 were treated by IV injection of 50 μmol/kg of CCXP1 targeted empty liposomes at day 15 and day 21. Mice from group 2 were treated by IV injection of CCXP1 targeted liposomes containing 100 μg of scFv-TM8-4-1BB-CD3z CAR mRNA and 50 μmol/kg liposomes at day 15 and day 21.

At day 25 mice were weighted and injected with 150 mg/kg luciferin. After 10 minutes of the luciferin administration, the animals were pre-anesthetized with the mixture gas of oxygen and isoflurane. When the animals were in a complete anesthetic state, bioluminescence measurements were performed with IVIS (Lumina II). After the luciferin test, the blood was sampled for polymorphonuclear leukocytes isolation and flow cytometry assessment.

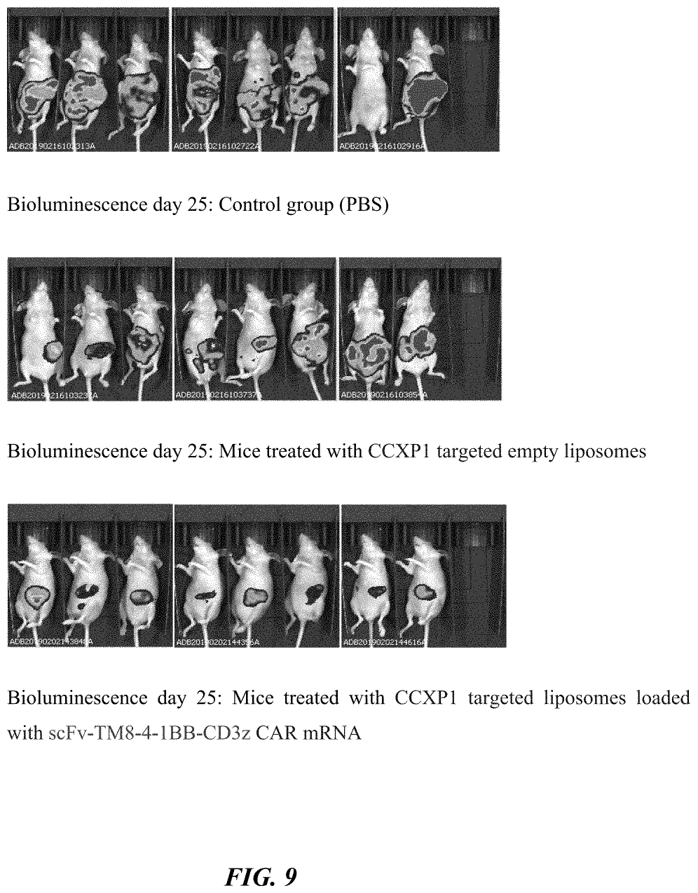

The bioluminescence data are shown in FIG. 9. The data clearly indicate that in vivo genetic reprogramming of polymorphonuclear leukocytes with injection of CCXP1-targeted liposomes loaded with scFv-TM8-4-1BB-CD3z CAR mRNA provides meaningful suppression of orthotopic pancreatic adenocarcinoma growths.

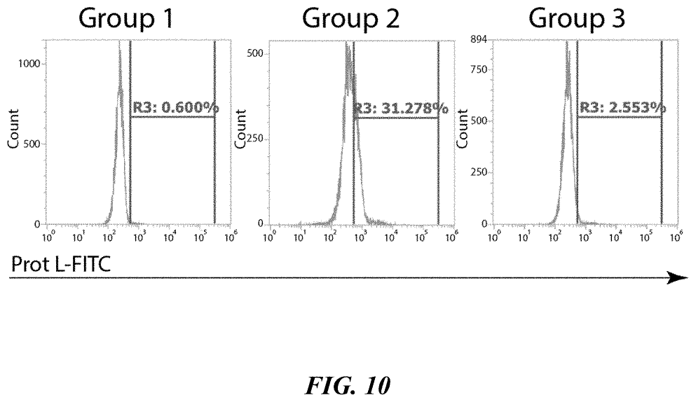

Expression of scFv-TM8-4-1BB-CD3z CAR in polymorphonuclear leukocytes was detected with flow cytometry using biotinylated protein L (Thermo Fisher Scientific) and streptavidin conjugated with FITC (Thermo Fisher Scientific). The data are shown in FIG. 10. The flow cytometry data clearly indicate that polymorphonuclear leukocytes genetically reprogrammed by CCXP1 targeted liposomes loaded with scFv-TM8-4-1BB-CD3z CAR mRNA in vivo.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating a cancer in a subject in need thereof comprising administering to the subject a therapeutically effective amount of polymorphonuclear leukocytes, wherein the polymorphonuclear leukocytes are genetically modified to express a recombinant chimeric antigen receptor (CAR) or T cell receptor (TCR).

2. The method of claim 1, wherein the polymorphonuclear leukocytes express a chimeric antigen receptor (CAR) targeting 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

3. The method of claim 1, wherein the polymorphonuclear leukocytes express a chimeric antigen receptor (CAR) targeting CD19.

4-5. (canceled)

6. The method of claim 1, wherein the polymorphonuclear leukocytes express a chimeric antigen receptor (CAR) targeting a human B cell receptor (BCR).

7-8. (canceled)

9. The method of claim 1, wherein the CAR or the TCR targets a tumor-specific peptide epitope.

10. The method of claim 1, wherein the polymorphonuclear leukocytes express at least two different CAR or TCR targeting at least two different epitopes.

11. The method of claim 1, wherein the polymorphonuclear leukocytes are genetically modified by mRNA transfection.

12-13. (canceled)

14. The method of claim 1, wherein the polymorphonuclear leukocytes are neutrophils.

15. (canceled)

16. A method of treating a cancer in a subject in need thereof comprising administering to the subject a therapeutically effective amount of a polynucleotide coding for a CAR or a TCR complexed with a carrier wherein the carrier comprises one or more molecules effective to deliver the polynucleotide to the cytoplasm of a polymorphonuclear leukocyte of the subject.

17-29. (canceled)

30. The method of claim 16, wherein the carrier is a recombinant histone H1 molecule.

31. The method of claim 30, wherein the recombinant histone H1 molecule is a human recombinant H1.3 molecule.

32. The method of claim 31, wherein the human recombinant H1.3 molecule additionally comprises an N-terminal bis-methionine sequence and the N,N-bismethionylhistone H1.3 molecule comprises the amino acid sequence of SEQ ID NO: 8.

33-38. (canceled)

39. A modified polymorphonuclear leukocyte, wherein the polymorphonuclear leukocyte expresses a recombinant chimeric antigen receptor (CAR) or T cell receptor (TCR).

40. The modified polymorphonuclear leukocyte of claim 39, wherein the polymorphonuclear leukocyte expresses a chimeric antigen receptor (CAR) targeting 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, mesothelin, IGF-1 receptor, IGF-I, IgGl, LI-CAM, IL-13, IL-6, insulin-like growth factor I receptor, integrin α5β1, integrin αvβ3, MORAb-009, MS4A, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin.

41-50. (canceled)

51. The modified polymorphonuclear leukocyte of claim 39, wherein the polymorphonuclear leukocyte expresses at least two different CAR or TCR targeting at least two different epitopes.

52-53. (canceled)
